(19) United States
(12) Patent Application Publication
(10) Pub. No.: US 2015/0190041 A1
(43) Pub. Date: Jul. 9, 2015

(54) ELONGATED MEMBER FOR MEDICAL USE AND CLEANING DEVICE

(71) Applicant: Terumo Kabushiki Kaisha, Tokyo (JP)

(72) Inventors: Satoru SUEHARA, Kaisei-machi (JP); Taiga NAKANO, Isehara-shi (JP); Hideyuki MOMOKI, Hadano-shi (JP)

(73) Assignee: Terumo Kabushiki Kaisha, Tokyo (JP)

(21) Appl. No.: 14/663,211

(22) Filed: Mar. 19, 2015

Related U.S. Application Data
(63) Continuation of application No. PCT/JP2013/074570, filed on Sep. 11, 2013.

(30) Foreign Application Priority Data
Sep. 28, 2012 (JP) 2012-215664

(51) Int. Cl. A61B 1/12; A61B 1/04; A61B 1/00; A61B 19/00

(52) U.S. Cl. CPC A61B 1/122; A61B 19/34; A61B 1/127; A61B 1/125; A61B 1/128; A61B 1/04; A61B 1/00163; A61B 2019/343

(57) ABSTRACT

An elongated member for medical use includes a lumen into which a medical instrument having an elongated main body section is insertable, the lumen being configured such that the medical instrument is advanceable and recedable through the lumen; and a cleaning unit configured to make contact with at least a portion of the medical instrument so as to clean the medical instrument when the medical instrument is advanced and receded through the lumen.

### CROSS-REFERENCE TO RELATED APPLICATIONS

This application is a continuation application filed under 35 U.S.C. 111(a) claiming the benefit under 35 U.S.C. §§120 and 365(c) of PCT International Application No. PCT/JP2013/074570 filed on Sep. 11, 2013, which is based upon and claims the benefit of priority of Japanese Application No. 2012-215664 filed on Sep. 28, 2012, the entire contents of which are hereby incorporated by reference in their entireties.

### BACKGROUND

The present disclosure relates to an elongated member for medical use that is used for cleaning a medical instrument and to a cleaning device including the elongated member.

In carrying out various procedures or treatments in living bodies, using an imaging device such as an endoscope to obtain images is commonly practiced. Such an imaging device normally includes an elongated main body section and a lens attached to the distal end (tip) of the main body section. In use, the main body section is pushed forward into the living body by a hand-side operation or the like while checking the image being picked up, whereby the distal end where the lens is disposed is guided toward a target part to be observed. In the use of an endoscope, when various body fluids, secretions or the like present in a living body are deposited on the lens, it may be impossible to obtain a clear image. It is desirable, therefore, that the matter deposited on the lens be cleaned or washed away upon each deposition. Also, in cases of using medical instruments other than the endoscope, such as a biopsy needle or biopsy forceps, deposition of body fluid, secretion or the like on various parts of the medical instrument may similarly occur during introduction of an elongated main body section into a living body. In these cases, also, it is desirable that the deposited matter be cleaned or washed away.

As a technology in connection with this, an endoscope device equipped with a wiper for cleaning a lens is disclosed in Japanese Patent Laid-Open No. 2012-86021. According to the disclosed technology, the wiper attached to a main body section of the endoscope is introduced into a living body together with the endoscope, whereby the lens of the endoscope can be cleaned inside the living body.

In the technology described in Japanese Patent Laid-Open No. 2012-86021, however, although cleaning of the lens is possible, there are some problems. An actuator for actuating the wiper must be provided on the hand operation side. Additionally, the wiper must be attached to the main body section distal end where the lens is disposed. Consequently, the device configuration of the endoscope becomes massive. In addition, it becomes difficult to realize an endoscope having a reduced diameter. Furthermore, the wiper system itself is low in convenience because it cannot be diverted to clean parts other than the lens of the endoscope or to clean medical instruments other than the endoscope.

### SUMMARY OF INVENTION

Disclosed here is an elongated member for medical use that, with a cleaning function, can be applied to a medical instrument having an elongated main body section, such as an endoscope, while preventing the device configuration of the medical instrument from becoming massive in scale and while preventing the medical instrument from being enlarged in diameter. Also disclosed here is a cleaning device including the elongated member.

Further disclosed is an elongated member for medical use that, with a cleaning function, can be diverted to clean parts other than a lens of an endoscope and to clean medical instruments other than the endoscope. Also disclosed is a cleaning device including the elongated member.

According to one embodiment, an elongated member for medical use includes: a lumen into and through which a medical instrument having an elongated main body section is inserted and passed in a manner of being able to advance and recede; and a cleaning unit that, attendantly on insertion and passage of the medical instrument into and through the lumen, makes contact with at least a portion of the medical instrument so as to clean the medical instrument.

Thus, the elongated member for medical use includes the cleaning unit that is simple in configuration and that cleans the medical instrument by making contact with the medical instrument attendantly on the insertion and passage of the medical instrument. Therefore, the elongated member for medical use can be applied to a medical instrument having an elongated main body section, such as an endoscope, while preventing the device configuration of the medical instrument from becoming massive in scale and while preventing the medical instrument from being enlarged in diameter. In addition, the elongated member for medical use can be diverted to clean parts other than the lens of an endoscope and to clean medical instruments other than the endoscope.

In one aspect, the cleaning unit is so disposed as to make contact with at least a distal portion of the medical instrument within the lumen.

According to this configuration, the cleaning unit makes contact with at least a distal portion of the medical instrument. Therefore, the distal portion and its periphery of the medical instrument can be cleaned in a reliable manner.

In one aspect, the cleaning unit further has a guide section that guides contact of the medical instrument with the cleaning unit.

This configuration ensures that the advancing and receding movements of the medical instrument in the lumen are guided by the guide section. Consequently, the advancing and receding movements of the medical instrument in the lumen are facilitated.

In one aspect, the cleaning unit includes at least one of a fabric, a brush, and a porous member.

When this configuration is adopted, the cleaning unit can be provided easily and inexpensively.

In one aspect, the elongated member for medical use further includes a supply/suction port that is provided in an inner wall surface of the lumen and through which a fluid is supplied into the lumen or a fluid in the lumen is sucked out.

In this case, because a fluid can be supplied into the lumen, it is possible, by supplying a cleaning liquid or compressed air into the lumen, to clean the medical instrument in a more favorable manner. Additionally, because a fluid in the lumen can be sucked out, it is possible, by sucking out body fluid present in the lumen, to prevent the deposition of the body fluid on the medical instrument.

In one aspect, the elongated member for medical use further includes a retention amount control unit that deforms at least a portion of the cleaning unit so as to reduce a liquid retention amount of the cleaning unit.

This configuration makes it possible to carry out dry wiping of the medical instrument. Accordingly, the medical instrument can be cleaned in a more sufficient manner.

In one aspect, the cleaning unit retains at least one of a cleaning liquid, an anti-fogging agent for preventing a transparent member from fogging, a hydrophilic agent, and a water repellent.

In this case, because at least one of a cleaning liquid, an anti-fogging agent for preventing a transparent member from fogging, a hydrophilic agent, and a water repellent is retained by the cleaning unit, it is possible to clean the medical instrument more sufficiently.

In one aspect, the elongated member for medical use is configured such that the cleaning unit includes at least a first contact member and a second contact member that each make contact with the medical instrument so as to clean the medical instrument, and the first contact member is disposed on an axially distal side of the second contact member and is composed of a member higher in tendency to retain a liquid than a member of which the second contact member is composed.

In this case, when the medical instrument is caused to advance and recede, “draining” and “coating” can be performed by the first contact member, and “scrubbing” can be conducted by the second contact member. Consequently, the medical instrument can be cleaned more satisfactorily.

In one aspect, the elongated member for medical use further includes a suction lumen through which a liquid is sucked out of the first contact member.

According to this configuration, it is possible to further reduce the amount of liquid retained by the first contact member. Therefore, dry wiping can be conducted with the first contact member in the state of retaining a more reduced amount of liquid. Consequently, the medical instrument can be cleaned more satisfactorily.

According to one embodiment, a cleaning device includes: the aforesaid elongated member for medical use; and a supply/suction unit that supplies a fluid to a cleaning unit of the elongated member or sucks a liquid out of the cleaning unit.

In this case, the liquid retention amount of, or the amount of liquid which is retained by, the cleaning unit can be controlled. Therefore, the medical device can be cleaned in a more favorable manner.

In one aspect, the fluid is at least one of air or a warmed fluid at a controlled temperature, a cleaning liquid, an anti-fogging agent for preventing a transparent member from fogging, a hydrophilic agent, and a water repellent.

Where the fluid is air, the cleaning device can be provided at a lower cost. Where the fluid is a warmed fluid at a controlled temperature, the liquid retention amount of the cleaning unit can be controlled so that the medical instrument can be cleaned more effectively. Where the fluid is a cleaning liquid, an anti-fogging agent for preventing a transparent member from fogging, a hydrophilic agent, or a water repellent, coating of the medical instrument can be conducted.

In the elongated member for medical use, in one aspect, the medical instrument is an imaging device that includes an imaging unit adapted to pick up an image and a lens.

When this configuration is adopted, the lens of the imaging device can be cleaned.

In the cleaning device as above, in one aspect, the medical instrument is an imaging device that includes an imaging unit adapted to pick up an image, and a lens.

When this configuration is adopted, the lens of the imaging device can be cleaned.

### BRIEF DESCRIPTION OF DRAWINGS

FIG. 1 is a schematic view showing a cleaning device according to a first embodiment of the present disclosure.

FIG. 2 is a side sectional view showing the cleaning device according to the first embodiment of the present disclosure.

FIG. 3 is a side sectional view showing the cleaning device in its state when an imaging device picks up an image according to the first embodiment.

FIG. 4 is a side sectional view showing a cleaning device according to a second embodiment of the present disclosure.

FIG. 5 is a sectional view taken along line 5-5 of FIG. 4.

FIG. 6 is a side sectional view showing a state where an imaging device picks up an image according to the second embodiment.

FIG. 7 is a side sectional view illustrating Modification 2, wherein one contact member is provided.

FIG. 8 is a side sectional view illustrating Modification, 3 wherein a second contact member is a brush oriented distally.

FIG. 9 is a side sectional view illustrating Modification 4, wherein a cleaning unit is a porous member.

FIG. 10 is a side sectional view illustrating Modification 5, wherein a cleaning unit has a spiral shape.

FIG. 11 is a side sectional view illustrating Modification 6, wherein an auxiliary section is provided between a cleaning unit and a lens.

FIG. 12 is a side sectional view illustrating Modification 7, wherein a cleaning section is provided in the periphery of an imaging device.

FIG. 13 is a side sectional view illustrating Modification 12, wherein a motor for rotating an imaging device is provided.

### DETAILED DESCRIPTION

### First Embodiment

Some embodiments, disclosed by way of example, of the present disclosure will now be described below. Note that for convenience of explanation, the dimensional ratios in the drawings may be exaggerated and be different from the actual ratios. In the following description, the hand operation side of a cleaning device 1 according to a first embodiment of the present disclosure will be referred to as the “proximal side,” and the side of insertion into a living-body lumen as the “distal side.”

A configuration of the cleaning device 1 according to the first embodiment of the present disclosure will be described.

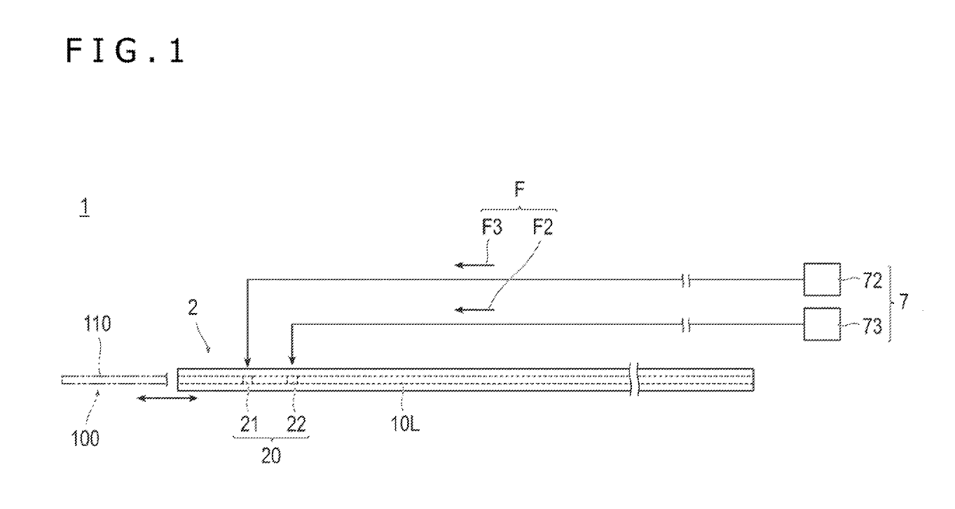

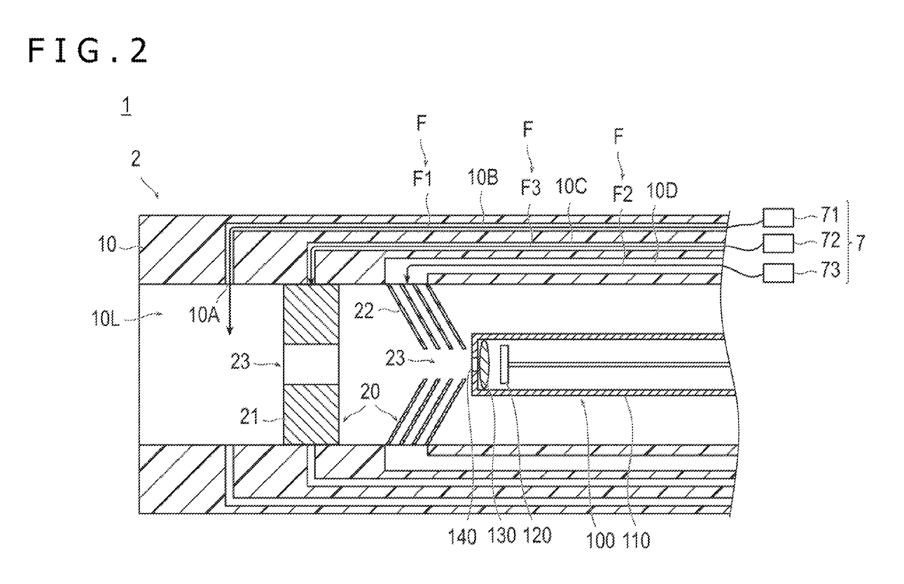

FIG. 1 is a schematic view showing the cleaning device 1 according to the first embodiment of the present disclosure, and FIG. 2 is a side sectional view showing the cleaning device 1 according to the first embodiment of the present disclosure.

The cleaning device 1 according to the first embodiment of the present disclosure will be outlined as follows. As shown in FIG. 1, the cleaning device 1 includes: an elongated member 2 for medical use having a lumen 10L into and through which an imaging device 100 including an elongated main body section 110 is inserted and passed in a manner of being able to advance and recede; and a cleaning unit 20 that, attendantly on the insertion and passage of the imaging device 100 into and through the lumen 10L, makes contact with a distal-side side surface of the imaging device 100 to clean the imaging device 100. The cleaning device 1 further includes a supply/suction unit 7 that supplies a fluid F to the cleaning unit 20 of the elongated member 2.

### Imaging Device 100

As shown in FIG. 2, the imaging device 100 includes the elongated main body section 110, an imaging unit 120 adapted to pick up an image, and a lens 130. The imaging device 100 further includes a light source (not shown) adapted to cast light on a subject.

The main body section 110 is provided, at a distal-side end surface thereof, with a light inlet part 140 through which the light from the subject is transmitted to the lens 130, and the lens 130 is disposed at the light inlet part 140.

The imaging device 120 is a two-dimensional image sensor such as Charge Coupled Device (CCD) or Complementary Metal Oxide Semiconductor (CMOS).

The lens 130 converges the light from the subject onto the imaging unit 120.

### Elongated Member 2

As depicted in FIG. 2, the elongated member 2 includes the main body section 10 being flexible and having a hollow elongated shape and the cleaning unit 20 adapted to clean the imaging device 100.

The main body section 10 defines the lumen 10L therein, and the imaging device 100 is inserted into and passed through the lumen 10L in the manner of being able to advance and recede. The main body section 10 includes: a supply/suction port 10A that is provided in an inner wall surface of the lumen 10L and through which a fluid F is supplied into the lumen 10L; a first lumen 10B that is formed in the main body section 10 and through which a gas F1 is supplied into the lumen 10L via the supply/suction port 10A; a second lumen 10C that is formed in the main body section 10 and through which a liquid F3 is supplied to a first contact member 21; and a third lumen 10D that is formed in the main body section 10 and through which a liquid F2 is supplied to a second contact member 22 of the cleaning unit 20 to be described later. Examples of the material constituting the main body section 10 include highly biocompatible materials such as thermoplastic resins, e.g., fluoro-resins such as tetrafluoroethylene-ethylene copolymer (ETFE), polytetrafluoroethylene (PTFE), etc., polyolefins such as polyethylene (PE), polypropylene (PP), etc., polyamides, polyesters, polyurethane, etc., and metals such as stainless steel, etc.

The supply/suction port 10A is a port through which the gas F1 is supplied from a first supply/suction unit 71 (described later) into the lumen 10L.

The first lumen 10B is a lumen through which the gas F1 is supplied from the first supply/suction unit 71 into the lumen 10L by way of the supply/suction port 10A.

The second lumen 10C is a lumen through which the liquid F3 is supplied from a second supply/suction unit 72 (described later) to the first contact member 21.

The third lumen 10D is a lumen through which the liquid F2 is supplied from a third supply/suction unit 73 (described later) to the second contact member 22.

The cleaning unit 20 is adapted to make contact with a distal-side end surface (distal portion) of the imaging device 100 so as to clean a surface of the lens 130 of the imaging device 100. The cleaning unit 20 includes: the first contact member 21 and the second contact member 22 that each make contact with the surface of the lens 130 so as to clean the surface of the lens 130; and a guide section 23 adapted to guide the contact of the imaging device 100 with the cleaning unit 20.

The first contact member 21 is disposed on an axially distal side of the second contact member 22 and is composed of a member higher in tendency to retain a liquid than a member of which the second contact member 22 is composed. The first contact member 21 is composed of a fabric. At the first contact member 21, “draining” and “coating” are conducted. “Draining” means to wipe off body fluid deposited on the surface of the lens 130 and to apply the liquid F3 to the surface of the lens 130. The first contact member 21 is fixed to the main body section 10 in a position corresponding to the second lumen 10C. Note that the method for fixation in this case is not specifically restricted. The fixation may be carried out, for example, by use of an adhesive, soldering, brazing, welding or the like.

The second contact member 22 is composed of a member lower in tendency to retain a liquid than the member of which the first contact member 21 is composed. The second contact member 22 is composed of a brush. At the second contact member 22, “scrubbing” is performed. The brush is configured to be oriented proximally. This ensures not only that the imaging device 100 can be easily moved from the distal side toward the proximal side but also that the area of contact of the surface of the lens 130 with the brush at the time of a distal movement of the imaging device 100 is increased, resulting in better cleaning of the surface of the lens 130, as compared with the case where the brush is not oriented proximally. The second contact member 22 is fixed to the main body section 10 in a position corresponding to the third lumen 10D.

The guide section 23 is composed of through-holes or slits provided in the vicinities of the centers of the first contact member 21 and the second contact member 22, respectively.

### Supply/Suction Unit 7

The supply/suction unit 7 supplies fluids F to the first contact member 21 and the second contact member 22 and into the lumen 10L. The supply/suction unit 7 includes the first supply/suction unit 71 adapted to supply the gas F1 into the lumen 10L through the first lumen 10B, the second supply/suction unit 72 adapted to supply the liquid F3 to the first contact member 21 via the second lumen 10C, and the third supply/suction unit 73 adapted to supply the liquid F2 to the second contact member 22 through the third lumen 10D.

The first supply/suction unit 71 supplies the gas F1 into the lumen 10L so as to remove debris, liquid and the like deposited on the surface of the lens 130. The gas F1 supplied from the first supply/suction unit 71 is preferably compressed air, but this is not limitative, and the gas F1 may be normal-pressure air, a warmed fluid at a controlled temperature, or the like. Note that while the first supply/suction unit 71 supplies the gas F1 into the lumen 10L in this embodiment, this is not restrictive, and the first supply/suction unit 71 may supply the liquid F2 such as a coating agent or a cleaning liquid. Though not illustrated, the first supply/suction unit 71 and the first lumen 10B are interconnected in a liquid-tight or gas-tight manner by use of a known tube.

The second supply/suction unit 72 supplies the liquid F3 to the first contact member 21. The liquid F3 supplied from the second supply/suction unit 72 is preferably a warmed fluid at a controlled temperature. Specific, but not limitative, examples of the liquid F3 are an anti-fogging agent for preventing a transparent member from fogging, a hydrophilic agent, and a water repellent. The liquid F3 may further be a surface modifying agent or may be a mixture of these liquids. In other words, a coating agent or the like is retained by the second contact member 22. This configuration is not restrictive, and the second supply/suction unit 72 may be able to supply normal-pressure air, compressed air, or the like. Note that though not illustrated, the second supply/suction unit 72 and the second lumen 10C are interconnected in a liquid-tight or gas-tight manner by a known tube.

The third supply/suction unit 73 supplies the liquid F2 to the second contact member 22. The liquid F2 supplied from the third supply/suction unit 72 is a cleaning liquid, but this is not limitative. The liquid F2 may be an anti-fogging agent for preventing a transparent member from fogging, a hydrophilic agent or a water repellent, or a mixture of these liquids. In other words, a cleaning liquid or the like is retained by the second contact member 22. Note that though not shown, the third supply/suction unit 73 and the third lumen 10D are interconnected in a liquid-tight or gas-tight manner by use of a known tube.

As aforementioned, the cleaning device 1 according to the first embodiment of the present disclosure has the elongated member 2 that includes the main body section 10 and the cleaning unit 20. The main body section 10 includes, formed therein: the lumen 10L; the supply/suction port 10A that is provided in the inner wall surface of the lumen 10L and through which a fluid F is supplied into the lumen 10L; the first lumen 10B through which the gas F1 is supplied into the lumen 10L via the supply/suction port 10A; the second lumen 10C that is provided on the axially proximal side of the first lumen 10B and through which the liquid F3 is supplied to the first contact member 21; and the third lumen 10D that is provided on the axially proximal side of the second lumen 10C and through which the liquid F2 is supplied to the second contact member 22. The cleaning unit 20 includes the first contact member 21 and the second contact member 22. The first contact member 21 is disposed on the axially distal side of the second contact member 22 and is composed of a member higher in tendency to retain a liquid than a member of which the second contact member 22 is composed.

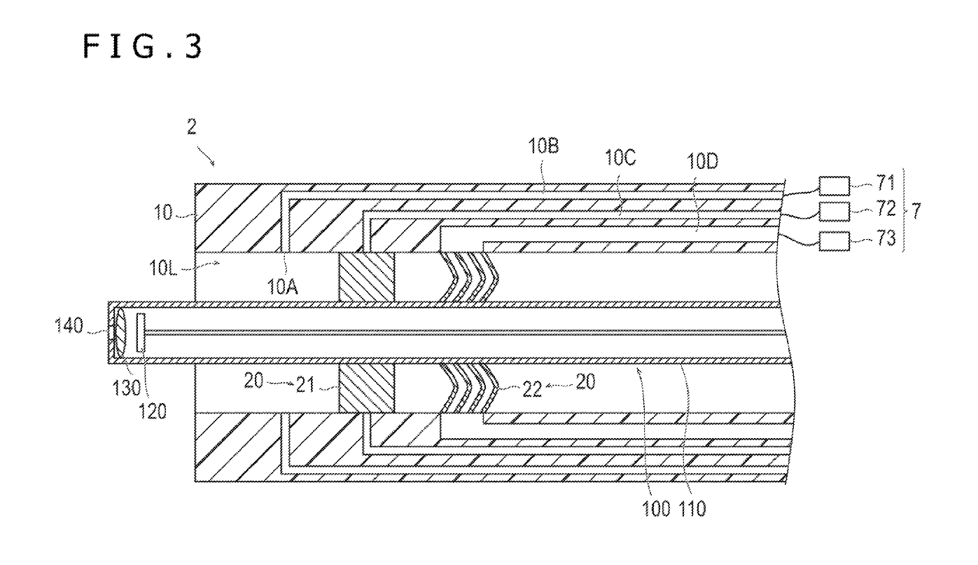

Now, an operation of the cleaning device 1 according to the first embodiment of the present disclosure will be described below referring to FIGS. 2 and 3. The following description will be made by taking, as an example, a case where it may be impossible, at the time of imaging by the imaging device 100, to pick up a clear image because body fluid present in a living body has been deposited on the surface of the lens 130. Note that the subject here may be, for example, a nasal cavity, an airway in a lung, a urethra, a stomach, or the like.

FIG. 3 is a side sectional view showing the cleaning device 1 in its state when an image is picked up by the imaging device 100 in the first embodiment. At the time of imaging by the imaging device 100, the imaging device 100 is pushed in distally in the axial direction, as shown in FIG. 3.

First, an operator supplies the liquid F2 from the third supply/suction unit 73 to the second contact member 22, to permit the liquid F2 to permeate the second contact member 22.

Next, the operator draws back the imaging device 100 proximally in the axial direction, as shown in FIG. 2.

In this step, the surface of the lens 130 comes into contact with the first contact member 21 and then the second contact member 22.

First, when the surface of the lens 130 passes by the supply/suction port 10A in the axial direction, the gas F1 is supplied from the first supply/suction unit 71 into the lumen 10L via the supply/suction port 10A, to partly remove the body fluid deposited on the surface of the lens 130. Then, the surface of the lens 130 makes contact with the first contact member 21, whereby body fluid deposited on the surface of the lens 130 is “drained” by the first contact member 21. Next, the surface of the lens 130 makes contact with the second contact member 22, whereby the surface of the lens 130 is “scrubbed” with the cleaning liquid or the like because the second contact member 22 is supplied with the liquid F2 by the third supply/suction unit 73.

Subsequently, the operator supplies the liquid F3 from the second supply/suction unit 72 to the first contact member 21, to permit the liquid F3 to permeate the first contact member 21 and, simultaneously, to wash away the body fluid that has been absorbed in the first contact member 21 during the drawing-back of the imaging device 100.

Next, the operator pushes in the imaging device 100 distally, as shown in FIG. 3.

In this step, the surface of the lens 130 comes into contact with the second contact member 22 and then the first contact member 21.

First, the surface of the lens 130 makes contact with the second contact member 22, thereby being “scrubbed” again. Next, the surface of the lens 130 makes contact with the first contact member 21, whereby “draining” and “coating” are performed. Further, when the surface of the lens 130 passes by the supply/suction port 10A in the axial direction, the gas F1 is supplied from the first supply/suction unit 71 into the lumen 10L, so that debris, liquid and the like deposited on the surface of the lens 130 are removed. Furthermore, a coating agent may be supplied from the first supply/suction unit 71 to be applied to the surface of the lens 130.

As aforementioned, according to the first embodiment of the present disclosure, the cleaning device 1 includes the cleaning unit 20, that has a simple configuration and that cleans the imaging device 100 by making contact with the imaging device 100 as the imaging device 100 is inserted and passed. Therefore, the cleaning device 1 can be applied to the imaging device 100 having the elongated main body section 110, while preventing the device configuration of the imaging device 100 from becoming massive in scale and while preventing the imaging device 100 from being enlarged in diameter. In addition, parts other than the lens, of the imaging device 100, can also be cleaned.

The cleaning unit 20 is disposed in the lumen 10L so as to make contact with a distal portion of the imaging device 100. This ensures reliable cleaning of the distal portion and its periphery of the imaging device 100.

The cleaning unit 20 has the guide section 23 adapted to guide the contact of the imaging device 100 with the cleaning unit 20. Therefore, the imaging device 100 is permitted to easily advance and recede in the lumen 10L.

The cleaning unit 20 is composed essentially of the fabric and the brush. This enables the guide section 23 to be formed easily and inexpensively.

The cleaning device 1 further includes the supply/suction port 10A that is provided in the inner wall surface of the lumen 10L and through which the fluid F is supplied into the lumen 10L. This ensures that by supplying compressed air or a cleaning liquid into the lumen 10L, the imaging device 100 can be cleaned in a more favorable manner.

At least one of a cleaning liquid, an anti-fogging agent for preventing a transparent member from fogging, a hydrophilic agent, and a water repellent is retained by the cleaning unit 20. Therefore, the surface of the lens 130 can be cleaned better.

The cleaning unit 20 includes the first contact member 21 and the second contact member 22 that each make contact with the imaging device 100 so as to clean the imaging device 100. The first contact member 21 is disposed on the axially distal side of the second contact member 22 and is composed of a member higher in tendency to retain a liquid than a member of which the second contact member 22 is composed. The imaging device 100 is caused to advance and recede, and therefore, the “draining” and the “coating” can be performed by the first contact member 21, and the “scrubbing” can be conducted by the second contact member 22. Accordingly, the surface of the lens 130 can be cleaned more assuredly.

The cleaning device 1 includes the supply/suction unit 7 adapted to supply the fluid F to the cleaning unit 20 of the elongated member 2. Therefore, the liquid retention amount of the first contact member 21 can be controlled, and the cleaning liquid can be supplied to the second contact member 22. Consequently, the surface of the lens 130 can be cleaned in a more favorable manner.

The fluid F is at least one of air or a warmed fluid at a controlled temperature, a cleaning liquid, an anti-fogging agent for preventing a transparent member from fogging, a hydrophilic agent, and a water repellent. Where the fluid F is air, the cleaning device 1 can be provided at a lower cost. Where the fluid F is a warmed fluid at a controlled temperature, the liquid retention amount of the first contact member 21 can be controlled, and therefore, the surface of the lens 130 can be cleaned more satisfactorily. Where the fluid F is a cleaning liquid, an anti-fogging agent for preventing a transparent member from fogging, a hydrophilic agent, or a water repellent, the surface of the lens 130 can be coated.

### Second Embodiment

A second embodiment of the present disclosure will be described below. In the following, descriptions of those features that are common to the first and second embodiments will be omitted, and features that are characteristic of only the second embodiment will be described.

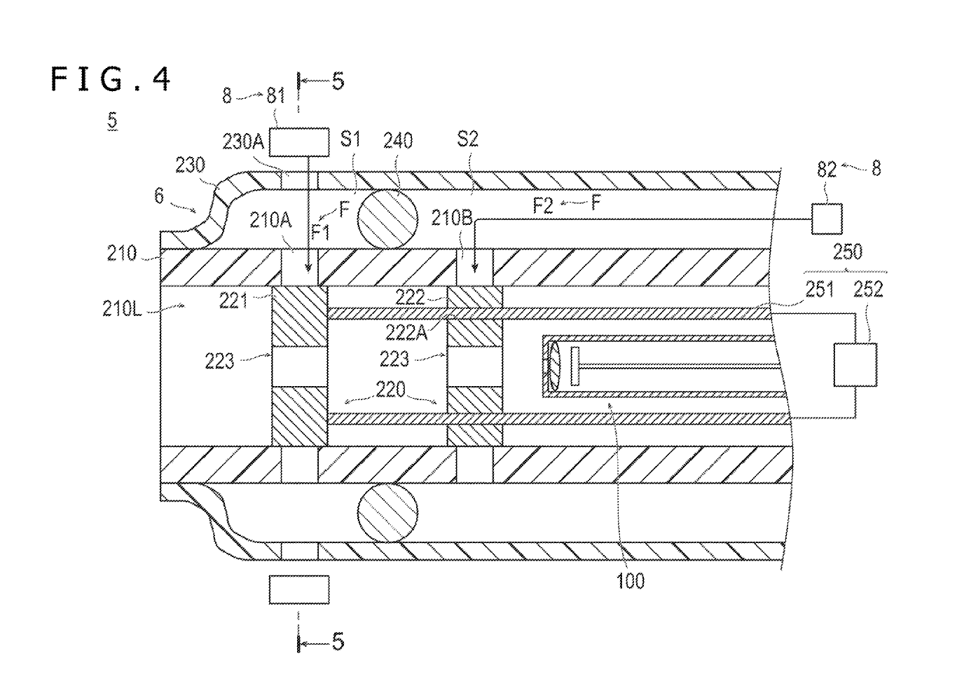

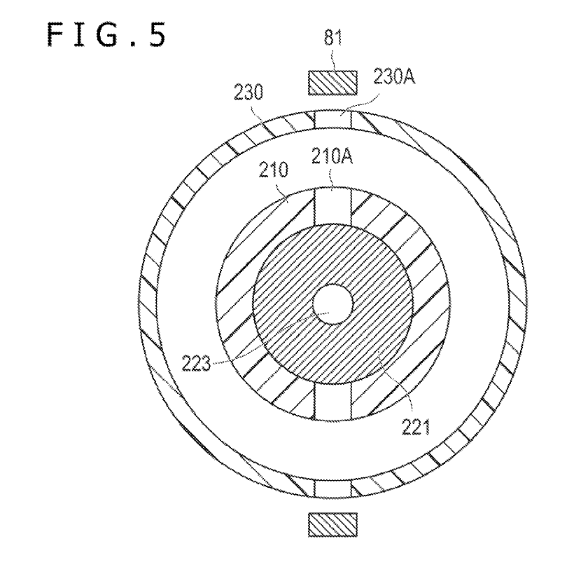

FIG. 4 is a side sectional view showing a cleaning device 5 according to the second embodiment of the present disclosure. FIG. 5 is a sectional view taken along line 5-5 of FIG. 4.

As shown in FIGS. 4 and 5, the cleaning device 5 according to the second embodiment of the present disclosure includes an elongated member 6 for medical use and a supply/suction unit 8. The elongated member 6 includes: a main body section 210 being flexible and having a hollow elongated shape; a cleaning unit 220 adapted to clean an imaging device 100; an outer tube 230 adapted to guide a fluid F to the cleaning unit 220; a seal section 240 adapted to control a flow of the fluid F; and a retention amount control unit 250 adapted to deform a first contact member 221 of the cleaning unit 220 so as to reduce the liquid retention amount of, or the amount of liquid which is retained by, the first contact member 221. The supply/suction unit 8 supplies the fluid F to the cleaning unit 220 of the elongated member 6.

The main body section 210 defines a lumen 210L therein, and the imaging device 100 is inserted into and passed through the lumen 210L in the manner of being able to advance and recede. The main body section 210 is formed with a first hole 210A in a position corresponding to the first contact member 221 of the cleaning unit 220 which will be described later and with a second hole 210B in a position corresponding to a second contact member 222 of the cleaning unit 220 which will be described later.

The cleaning unit 220 makes contact with a distal-side end surface (distal portion) of the imaging device 100 so as to clean a surface of a lens 130. The cleaning unit 220 includes the first contact member 221 and the second contact member 222 that each make contact with the surface of the lens 130 so as to clean the surface of the lens 130.

The first contact member 221 is disposed on the axially distal side of the second contact member 222 and is made, under the function of the supply/suction unit 8, to retain less liquid than the second contact member 222 does. Therefore, at the first contact member 221, “dry wiping” is performed. The first contact member 221 is fixed to the main body section 210 in such a manner as to close the first hole 210A. The first contact member 221 is composed of a fabric.

The second contact member 222 is configured to retain more liquid than the first contact member 221 does. Therefore, at the second contact member 222, “wet wiping” is conducted. The second contact member 222 is provided therein with a third hole 222A configured to prevent an interlock portion 251 of the retention amount control unit 250 from interfering with the second contact member 222 when rotated. The second contact member 222 is fixed to the main body section 210 in such a manner as to close the second hole 210B. The second contact member 222 is composed of a fabric.

The outer tube 230 guides the fluid F to the cleaning unit 220. The outer tube 230 is fixed to the main body section 210 on a distal side and, together with the seal section 240, defines a first space S1 on the distal side of the seal section 240 and a second space S2 on a proximal side of the seal section 240. The outer tube 230 is provided with a fourth hole 230A through which a gas F1 supplied from a gas supplying section 81 of the supply/suction unit 8 described later is to be introduced into the first space S1. Examples of the material constituting the outer tube 230 include highly biocompatible materials such as thermoplastic resins, e.g., fluoro-resins such as tetrafluoroethylene-ethylene copolymer (ETFE), polytetrafluoroethylene (PTFE), etc., polyolefins such as polyethylene (PE), polypropylene (PP), etc., polyamides, polyesters, polyurethane, etc.

The seal section 240 offers a seal such that the fluids F introduced respectively into the first space S1 and the second space S2 will not mix with each other. The seal section 240 may be, for example, an O-ring-like seal member. In this instance, the outside diameter of the seal section 240 is set to be smaller than the width defined by the main body section 210 and the outer tube 230.

The retention amount control unit 250 reduces the liquid retention amount of, or the amount of liquid which is retained by, the first contact member 221. The retention amount control unit 250 includes: the interlock portion 251 that is fixed to the first contact member 221 and that is higher in rigidity than the first contact member 221; and a rotary section 252 adapted to rotate the interlock portion 251.

The interlock portion 251 is fixed to a right end portion of the first contact member 221, is passed through the third hole 222A, and is extended proximally. Note that the method for fixation here is not specifically restricted. The fixation may be conducted, for example, by use of an adhesive, soldering or the like. The interlock portion 251 may be formed from metal, for example. The material of the interlock portion 251, however, is not limited to metal but may be resin or the like.

The rotary section 252 is connected to the interlock portion 251 on a proximal side, and rotates the interlock portion 251. The rotary section 252 may be a motor, for example. When the rotary section 252 rotates, the first contact member 221 is rotated by way of the interlock portion 251, whereby the amount of liquid that is retained by the first contact member 221 can be reduced. Note that while the interlock portion 251 is automatically rotated by the rotary section 252, the interlock portion 251 may be rotated manually.

The supply/suction unit 8 supplies the fluid F1 to the first contact member 221 and the fluid F2 to the second contact member 222. The supply/suction unit 8 includes: the gas supplying section 81 that is provided around the periphery of the fourth hole 230A of the outer tube 230 and supplies the gas F1 to the first contact member 221; and a liquid supplying section 82 that supplies a liquid F2 to the second contact member 222.

The gas supplying section 81 supplies the gas F1 to the first contact member 221 so as to reduce the liquid retention amount of the first contact member 221. Specifically, the gas F1 supplied from the gas supplying section 81 is introduced into the first space S1 via the fourth hole 230A and is further passed through the first hole 210A to be supplied to the first contact member 221. In this instance, the seal section 240 prevents the gas F1 from moving from the first space S1 into the second space S2.

The liquid supplying section 82 supplies the liquid F2 to the second contact member 222. Specifically, the liquid F2 supplied from the liquid supplying section 82 is introduced into the second space S2 and moves through the second space S2 from the proximal side toward the distal side to be supplied through the second hole 210B to the second contact member 222. In this instance, the seal section 240 prevents the liquid F2 from moving from the second space S2 into the first space S1.

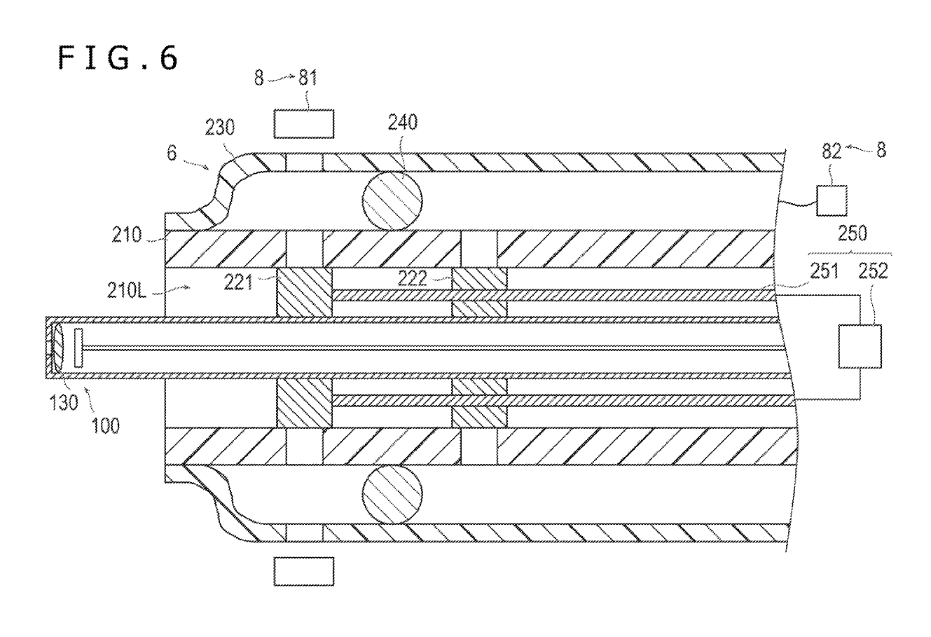

Now, an operation of the cleaning device 5 according to the second embodiment of the present disclosure will be described, referring to FIGS. 4 and 6. The following description will be made by taking as an example a case where it may be impossible, at the time of imaging by the imaging device 100, to pick up a clear image because body fluid present in a living body has been deposited on the surface of the lens 130. Note that the subject here may be, for example, a nasal cavity, an airway in a lung, a urethra, a stomach, or the like.

FIG. 6 is a side sectional view showing the cleaning device 5 in its state when an image is picked up by the imaging device 100. At the time of imaging by the imaging device 100, the imaging device 100 is pushed in distally in the axial direction, as shown in FIG. 6.

First, an operator supplies the gas F1 from the gas supplying section 81 to the first contact member 221, thereby reducing the liquid retention amount of the first contact member 221. In addition, the operator supplies the liquid F2 from the liquid supplying section 82 to the second contact member 222, to permit the liquid F2 to permeate the second contact member 222.

Next, the operator draws back the imaging device 100 proximally in the axial direction, as shown in FIG. 4.

In this step, the surface of the lens 130 comes into contact with the first contact member 221 and then the second contact member 222.

In this case, first, the surface of the lens 130 makes contact with the first contact member 221, whereby body fluid deposited on the surface of the lens 130 is absorbed by the first contact member 221 having a reduced liquid retention amount. Then, the surface of the lens 130 makes contact with the second contact member 222. Because the second contact member 222 has been supplied with the liquid F2 by the liquid supplying section 82, the surface of the lens 130 is thus subjected to “wet wiping” with the cleaning liquid retained by the second contact member 222.

Subsequently, the operator operates the rotary section 252, whereby the first contact member 221 impregnated with the body fluid is rotated via the interlock portion 251, and the body fluid is squeezed out. Even during this process, the supply of the gas F1 from the gas supplying section 81 to the first contact member 221 is being continued so that the body fluid having permeated the first contact member 221 is vaporized. As a result, the liquid retention amount of the first contact member 221 is reduced.

Next, the operator pushes in the imaging device 100 distally, as shown in FIG. 6.

In this step, the surface of the lens 130 comes into contact with the second contact member 222 and then the first contact member 221.

In this case, first, the surface of the lens 130 makes contact with the second contact member 222, thereby being “wet wiped” again. Then, the surface of the lens 130 makes contact with the first contact member 221, whereby the surface is “dry wiped” because the first contact member 221 is retaining a reduced amount of liquid, as aforementioned.

As described above, according to the second embodiment of the present disclosure, the cleaning device 5 includes the cleaning unit 220 that has a simple configuration and cleans the imaging device 100 by making contact with the imaging device 100 as the imaging device 100 is inserted and passed. Therefore, the cleaning device 5 can be applied to the imaging device 100 having the elongated main body section 110, while preventing the device configuration of the imaging device 100 from becoming massive in scale and while preventing the imaging device 100 from being enlarged in diameter. In addition, parts other than the lens of the imaging device 100 can also be cleaned.

The cleaning device 5 further has the retention amount control unit 250 that deforms the first contact member 221 of the cleaning unit 220 so as to reduce the liquid retention amount of, or the amount of liquid which is retained by, the first contact member 221. Therefore, the surface of the lens 130 can be dry wiped by the first contact member 221. Consequently, the surface of the lens 130 can be cleaned more satisfactorily.

### Modifications

Now, modifications will be shown below by way of example.

### Modification 1

In the first and second embodiments described above, the supply/suction unit 7 and 8 supplies the fluid F to the first contact member 21 and 221 and the second contact member 22 and 222 and into the lumen 10L, and the supply/suction port 10A supplies the fluid F into the lumen 10L therethrough. However, the supply/suction unit 7 and 8 may suck the liquid out of the first contact member 21 and 221, the second contact member 22 and 222 and the lumen 10L, and the supply/suction port 10A may serve for sucking the liquid out of the lumen 10L therethrough. For instance, the second lumen 10C in the first embodiment may be used as a suction lumen for sucking out a liquid therethrough, and the second supply/suction unit 72 may suck the liquid out of the first contact member 21, whereby the amount of liquid retained by the first contact member 21 can be reduced. This ensures that dry wiping can be carried out in the condition where the first contact member 21 is retaining a more reduced amount of liquid, so that the surface of the lens 130 can be cleaned more satisfactorily.

### Modification 2

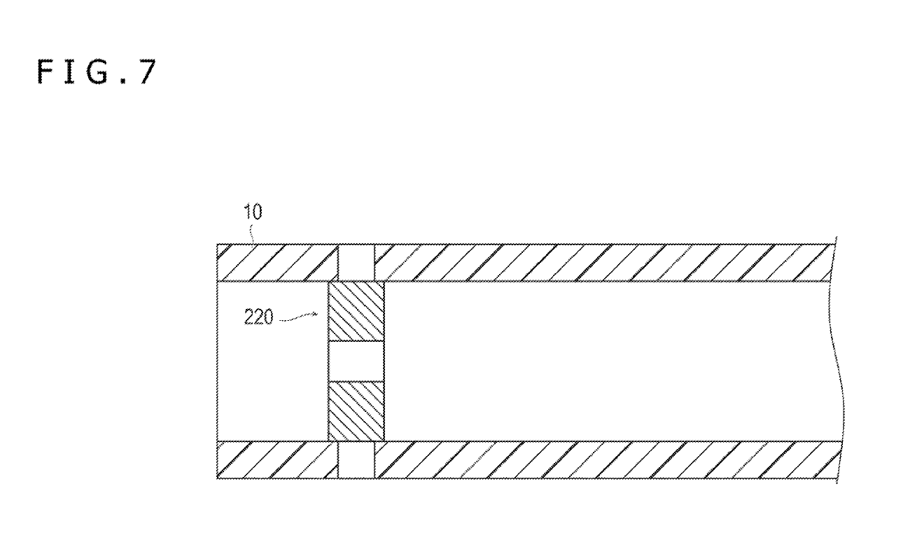

FIG. 7 is a side sectional view illustrating Modification 2. In the first and second embodiments described above, the cleaning unit 20 and 220 has two contact members, namely, the first contact member 21 and 221 and the second contact member 22 and 222. However, only one contact member may be provided, as shown in FIG. 7. Alternatively, three or more contact members may be provided.

### Modification 3

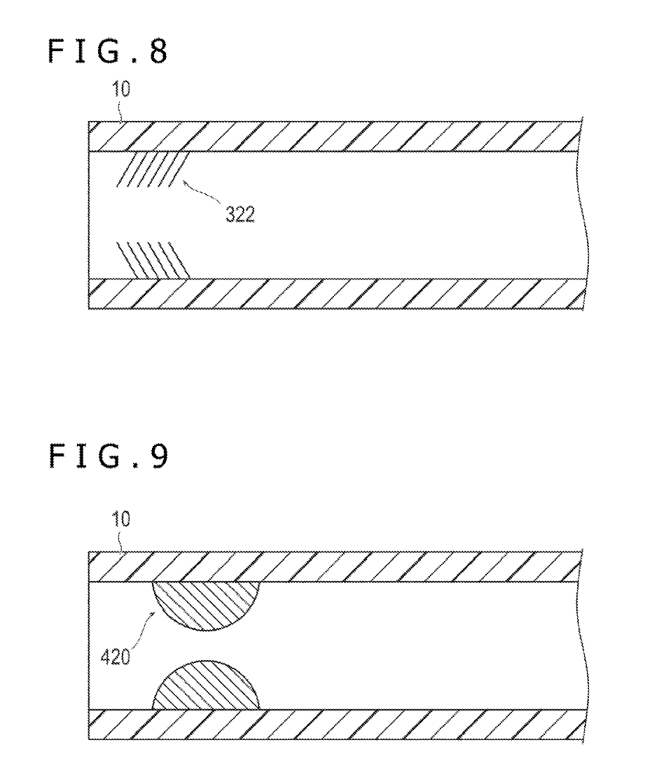

FIG. 8 is a side sectional view illustrating Modification 3. In the first embodiment described above, the second contact member 22 of the cleaning unit 20 has a configuration in which a brush is oriented proximally. However, there may be adopted a second contact member 322 having a configuration in which the brush is oriented distally, as depicted in FIG. 8.

### Modification 4

FIG. 9 is a side sectional view illustrating Modification 4. In the first and second embodiments described above, the cleaning unit 20 and 220 is configured by use of a fabric and/or a brush. However, there may be adopted a cleaning unit 420 configured by use of a porous member, as shown in FIG. 9. According to this configuration, the surface of the lens 130 can be cleaned more favorably because the porous member permits an easy control of the liquid retention amount thereof.

### Modification 5

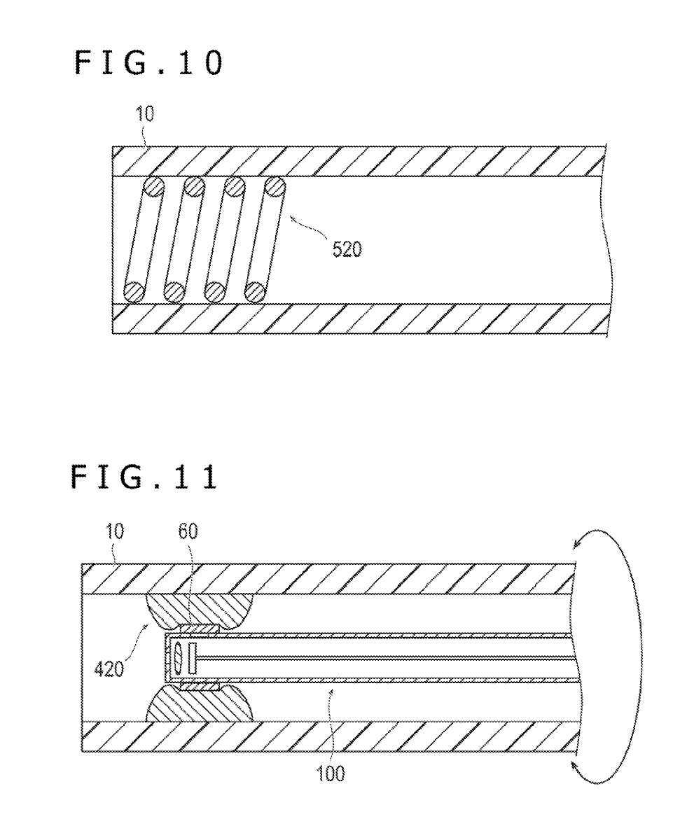

FIG. 10 is a side sectional view illustrating Modification 5. In the second embodiment described above, the cleaning unit 220 has a hollow cylindrical shape as depicted in FIG. 5. However, there may be adopted a cleaning unit 520 that has a spiral shape, as shown in FIG. 10. According to this configuration, the surface of the lens 130 can be cleaned more sufficiently because the cleaning unit 520 has projections and recesses along the advancing and retracting direction of the imaging device due to the spiral shape.

### Modification 6

FIG. 11 is a side sectional view illustrating Modification 6. In the second embodiment described above, the first contact member 221 is deformed by the retention amount control unit 250, whereby the liquid retention amount of the first contact member 221 is reduced. Where a cleaning unit 420 composed of a porous member as shown in FIG. 11 is adopted, however, an auxiliary section 60 higher in rigidity than the cleaning unit 420 may be provided between the cleaning unit 420 and the lens 130, and the main body section 10 may be rotated so that the auxiliary section 60 presses the cleaning unit 420, thereby reducing the amount of liquid that is retained by the cleaning unit 420.

### Modification 7

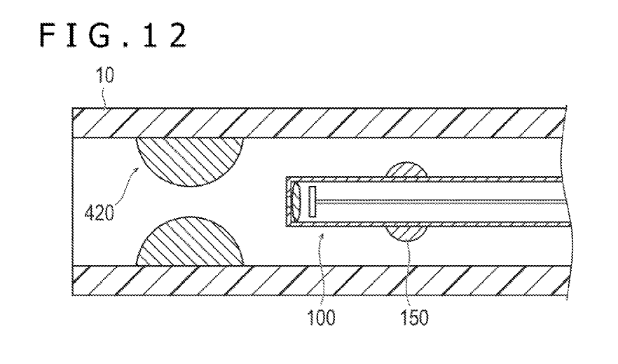

FIG. 12 is a side sectional view illustrating Modification 7. As shown in FIG. 12, a cleaning section 150 for cleaning a cleaning unit 420 may be provided in the periphery of the imaging device 100. According to this configuration, as the imaging device 100 is inserted and passed, the cleaning section 150 makes contact with the cleaning unit 420, whereby the cleaning unit 420 can be cleaned.

### Modification 8

In the second embodiment described above, the retention amount control unit 250 is interlocked only to the first contact member 221. However, the retention amount control unit 250 may be interlocked only to the second contact member 222 or may be interlocked to both the first contact member 221 and the second contact member 222.

### Modification 9

In the first and second embodiments described above, the imaging device 100 is taken as an object to be cleaned by the cleaning device 1 and 5. However, the object to be cleaned by the cleaning device 1 and 5 may be a sensor such as a gas sensor, an ultrasonic sensor, etc., a laser for use in laser therapy, a probe or the like. Thus, the elongated member 2 and 6 and the cleaning device 1 and 5 according to this embodiment can be diverted to cleaning of medical instruments other than the imaging device.

### Modification 10

In the first and second embodiments described above, a two-dimensional image sensor such as CCD or CMOS is mentioned as the imaging unit 120 possessed by the imaging device 100 for picking up an image. However, the imaging unit 120 may be an imaging element having a structure in which optical fibers are used.

### Modification 11

In the first embodiment described above, the guide section 23 has a predetermined size such that the imaging device 100 can be easily inserted and passed therein, as shown in FIG. 2. However, the guide section 23 may be configured in a smaller size. This configuration enables more assured cleaning of the whole distal-side surface of the imaging device 100.

### Modification 12

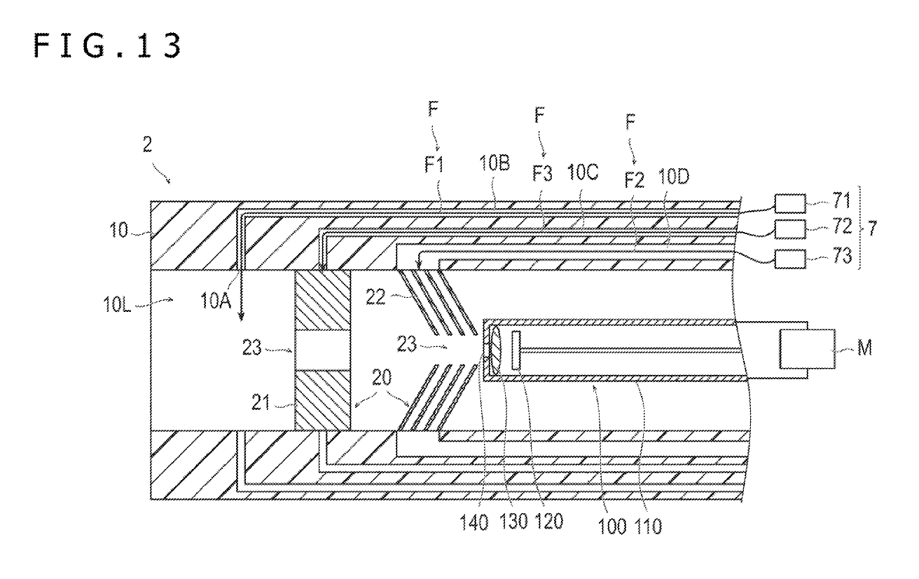

FIG. 13 is a side sectional view illustrating Modification 12. As shown in FIG. 13, a motor M for rotating the imaging device 100 may further be provided. According to this configuration, the imaging device 100 is brought into contact with the first contact member 21 and the second contact member 22 while being rotated by the motor M. Therefore, the surface of the lens 130 can be cleaned more favorably. Note that, though not shown, a motor or motors for rotating the first contact member 21 and/or the second contact member 22 may further be provided. Furthermore, a configuration may be adopted in which the imaging device 100, the first contact member 21, and the second contact member 22 are rotated manually.

It should be understood by those skilled in the art that various modifications, combinations, sub-combinations and alterations may occur depending on design requirements and other factors insofar as they are within the scope of the appended claims or the equivalents thereof.

What is claimed is:

1. An elongated member for medical use, comprising:
a lumen into which a medical instrument having an elongated main body section is insertable, the lumen being configured such that the medical instrument is advanceable and recedable through the lumen; and
a cleaning unit configured to make contact with at least a portion of the medical instrument so as to clean the medical instrument when the medical instrument is advanced and receded through the lumen.

2. The elongated member for medical use according to claim 1, wherein the cleaning unit is configured to make contact with at least a distal portion of the medical instrument within the lumen.

3. The elongated member for medical use according to claim 1, wherein the cleaning unit includes a guide section configured to guide the contact of the medical instrument with the cleaning unit.

4. The elongated member for medical use according to claim 1, wherein the cleaning unit includes at least one of a fabric member, a brush member, and a porous member.

5. The elongated member for medical use according to claim 1, further comprising a supply/suction port that is located in an inner wall surface of the lumen and through which a fluid is suppliable into or suckable from the lumen.

6. The elongated member for medical use according to claim 1, further comprising a retention amount control unit configured to deform at least a portion of the cleaning unit so as to reduce a liquid retention amount of the cleaning unit.

7. The elongated member for medical use according to claim 1, wherein the cleaning unit is configured to retain at least one of a cleaning liquid, an anti-fogging agent, a hydrophilic agent, and a water repellent.

8. The elongated member for medical use according to claim 1,
wherein the cleaning unit includes a first contact member and a second contact member that are each configured to make contact with the medical instrument so as to clean the medical instrument, and
wherein the first contact member is disposed on an axially distal side of the second contact member and is composed of a member higher in tendency to retain a liquid than a member of which the second contact member is composed.

9. The elongated member for medical use according to claim 1, wherein the medical instrument is an imaging device that includes:
an imaging unit configured to pick up an image, and
a lens.

10. The elongated member for medical use according to claim 4, further comprising a supply/suction port that is located in an inner wall surface of the lumen and through which a fluid is suppliable into or suckable from the lumen.

11. The elongated member for medical use according to claim 7, further comprising a supply/suction port that is located in an inner wall surface of the lumen and through which a fluid is suppliable into or suckable from the lumen.

12. The elongated member for medical use according to claim 8, further comprising a suction lumen through which a liquid is suckable out of the first contact member.

13. The elongated member for medical use according to claim 8, further comprising a supply/suction port that is located in an inner wall surface of the lumen and through which a fluid is suppliable into or suckable from the lumen.

14. A cleaning device comprising:
an elongated member for medical use, including:
a lumen into which a medical instrument having an elongated main body section is insertable, the lumen being configured such that the medical instrument is advanceable and recedable through the lumen; and
a cleaning unit configured to make contact with at least a portion of the medical instrument so as to clean the medical instrument when the medical instrument is advanced and receded through the lumen; and
a supply/suction unit that is configured to supply a fluid to a cleaning unit of the elongated member or suck a fluid out of the cleaning unit.

15. The cleaning device according to claim 14, wherein the fluid is at least one of air, a warmed fluid at a controlled temperature, a cleaning liquid, an anti-fogging agent, a hydrophilic agent, and a water repellent.

16. The cleaning device according to claim 14, wherein the medical instrument is an imaging device that includes:
an imaging unit configured to pick up an image, and
a lens.

17. The cleaning device according to claim 14, wherein the cleaning unit includes at least one of a fabric member, a brush member, and a porous member.

18. The cleaning device according to claim 14, wherein the cleaning unit is configured to retain at least one of a cleaning liquid, an anti-fogging agent, a hydrophilic agent, and a water repellent.

19. The cleaning device according to claim 14,
wherein the cleaning unit includes a first contact member and a second contact member that are each configured to make contact with the medical instrument so as to clean the medical instrument, and
wherein the first contact member is disposed on an axially distal side of the second contact member and is composed of a member higher in tendency to retain a liquid than a member of which the second contact member is composed.
